(19) United States
(12) Patent Application Publication
(10) Pub. No.: US 2009/0118262 A1
(43) Pub. Date: May 7, 2009

(54) Non-Aqueous Water-Miscible Materials as Vehicles for Drug Delivery

(76) Inventors: Brian R. Rohrs, Fairport, NY (US); Martin J. Coffey, Pittsford, NY (US)

Correspondence Address: Bausch & Lomb Incorporated, One Bausch & Lomb Place, Rochester, NY, 14604-2701, US

(21) Appl. No.: 12/253,341

(22) Filed: Oct. 17, 2008

Related U.S. Application Data
(60) Provisional application No. 60/984,405, filed on Nov. 1, 2007.

(51) Int. Cl. A61K 31/55; A61K 31/47; A61P 27/02

(52) U.S. Cl. 514/217.07; 514/311

(57) ABSTRACT

A pharmaceutical composition includes at least one pharmaceutical component having a low aqueous solubility and at least one non-aqueous water-miscible material. Such a pharmaceutical composition is useful in providing a therapeutically meaningful amount of such pharmaceutical component at a target tissue. The pharmaceutical composition is particularly suitable for administration to or into an ocular environment to treat or control an ocular disease, disorder, or condition.

### CROSS REFERENCE

This application claims the benefit of U.S. Provisional Patent Application No. 60/984,405 filed Nov. 1, 2007 which is incorporated by reference herein.

### FIELD OF THE INVENTION

The present invention relates to pharmaceutical compositions and pharmaceutical kits comprising at least one pharmaceutical component that is insoluble or poorly soluble in water and a non-aqueous water-miscible material. In particular, the present invention relates to the use of such compositions for effective delivery of therapeutic amounts of such pharmaceutical component to target tissues of a human or animal. In particular, such target tissues are ocular tissues. Methods of treatment utilizing such pharmaceutical compositions are also envisaged.

### BACKGROUND OF THE INVENTION

Many pharmaceutical components are insoluble or have low solubility in water making them difficult to be formulated into pharmaceutical compositions that can provide therapeutically effective amounts of the pharmaceutical components at or to the targeted tissues of a human or animal.

In general, the outer elements of the eye comprise the lacrimal apparatus and the conjunctival sac. The eye also includes a number of other structures. For example, the sclera serves as the outer coating of the eyeball while a colored membrane called the iris regulates the entrance of light through the pupil, a contractile opening at the center of the iris that responds to light and darkness. The lens of the eye is a transparent refracting body that focuses light rays to form an image on the retina, which in turn receives and transmits them to the brain via the optic nerve. To nourish such structures and to assist with the removal of waste products, the aqueous humor, a fluid derived from the blood by a process of secretion and ultrafiltration through the ciliary processes circulates from the posterior chamber to the anterior chamber of the eye and leaves the eye through the trabecular network and Schlemm's canal. Lastly, eyelids and a mucous membrane that lines the eyelids known as the conjunctiva protect the eye and distribute tears. Thus, in light of such structural differentiation, the delivery of therapeutic ophthalmic components to the ocular environment can be very challenging.

Topical application is the most common route of administration of ophthalmic components. Advantages of such an application can include convenience, simplicity, noninvasive nature, and the ability of the patient to self-administer. For example, most topical ocular preparations are commercially available as solutions or suspensions that are applied directly to the eye via an applicator such as an eye dropper.

U.S. Pat. No. 5,480,914 and U.S. Pat. No. 5,620,699, both to Meadows, describe drop-instillable topical, nonaqueous thixotropic drug delivery vehicles containing a substantially homogeneous dispersion of at least one suspending aid in a nonaqueous perflourocarbon or fluorinated silicone liquid carrier for use in delivering ophthalmic components to aqueous physiological systems such as the eye. U.S. Pat. No. 3,767,788 to Rankin describes a drop-instillable ophthalmic solution containing an aqueous solution of polyethylene oxide, optionally polyethylene glycol, and other optional ingredients to lubricate and cushion eyes traumatized by contact lens wear.

Alternatively, ophthalmic components may be delivered topically to the eye via an ointment or gel. Such delivery vehicles prolong contact time with the external ocular surface and can offer extended dosing intervals such as “sustained release” type dosing. Ophthalmic components may also be delivered topically to the eye by devices such as contact lenses, cotton pledgets, or membrane-bound inserts.

Soft contact lenses can absorb water-soluble drugs and release them to the eye over prolonged periods of time whereas cotton pledgets (i.e., small pieces of cotton) can be saturated with ophthalmic solutions and placed in the conjunctival sac to topically deliver medicaments. A membrane-bound insert (e.g., Ocusert®) is a membrane-controlled drug delivery system. Following placement onto the bulbar conjunctiva under the upper or lower eyelid, the device releases ophthalmic medicaments slowly over time.

However, because of losses of the administered ophthalmic formulation through tear drainage, topically administered medicaments do not typically penetrate in useful concentrations to the posterior cavity of the eye, and therefore, are of little therapeutic benefit to treat or control diseases of the retina, optic nerve and other posterior segment structures. Additionally, some currently available topical delivery vehicles themselves have inherent disadvantages. For example, ointments may impede delivery of other ophthalmic components by serving as a barrier to contact. Ointments may also blur vision after administration. Moreover, the efficacy of ophthalmic medicaments in suspension, which are delivered via drop applicators, can be inconsistent due to easy settlement of the active ingredients from the suspension. As a result, proper administration technique frequently determines the efficacy of such medicaments.

Formulating techniques can also play a significant role in drug delivery and therapeutic outcomes in the ocular environment. Several ophthalmic components are poorly soluble in a variety of topical drug delivery vehicles, in turn, making delivery to the posterior cavity in an efficacious manner difficult. To overcome such difficulties associated with topical administration, ophthalmic components can be delivered to regions of the posterior cavity via ocular injection routes of administration. Thus, a number of ocular injection methodologies have been employed to deliver ophthalmic components.

U.S. Pat. No.5,718,922 to Herrero-Vanrell et al., describes a method of forming microspheres containing a hydrophilic drug or agent for injection within the eye to provide localized treatment over a sustained period of time. Alternatively, U.S. Pat. No. 5,336,487 to Refojo et al., describes a method of treating an intraocular structural disorder of the retina by injecting a liquid silicone/fluorosilicone oil emulsion into the vitreous humor of the eye to treat the disorder and allow the retina to heal. However, such microspheres or emulsions may occlude the visual axis when delivered by an intravitreal injection.

Alternatively, U.S. Pat. No. 5,366,739 and U.S. Pat. No. 5,830,508, both to MacKeen, describe a composition and method for topical, prolonged delivery of a therapeutic agent to the eye for the treatment of dry eye syndrome. The therapeutic agent is further described as a water-soluble, calcium-based composition that is placed within a carrier, which is preferably hydrophobic/non-aqueous in nature (e.g., petrolatum or a combination of petrolatum and white wax). The composition is then delivered manually or by sterile cotton application to the extraocular skin adjacent to the lateral canthus of the eye. Although non-aqueous delivery vehicles are described for topical application for extraocular usage, injectable compositions and methods are not disclosed.

Further, a review of solubilizing excipients for oral and injectable formulations by Robert G. Strickley describes such agents as including water-soluble solvents (e.g., polyethylene glycol 300), non-ionic surfactants (polysorbate 80), water-soluble lipids (e.g., castor oil), organic liquids/semi-solids (e.g., beeswax), and various cyclodextrins and phospholipids. See R. G. Strickley, Solubilizing Excipients in Oral and Injectable Formulations, Pharmaceutical Research, Vol. 21, No. 2, pp. 201-30 (February 2004). However, ocular injectable formulations, especially extended, controlled or sustained release-based formulations for injection into the posterior regions of the ocular environment are not disclosed.

As discussed above, delivering therapeutic compounds to the ocular environment can be challenging. Therefore, while medicaments are currently available to treat ocular diseases, there still is a need for improved ophthalmic compositions and methods for delivering such compositions to the posterior regions of the ocular environment, especially to achieve an extended, controlled or sustained release of the active ingredients of such compositions. Novel and improved compositions can significantly overcome existing difficulties in providing therapeutically effective amounts of the pharmaceutical components to the targeted tissues.

### SUMMARY OF THE INVENTION

In general, the present invention provides pharmaceutical compositions, pharmaceutical kits, and methods of treatment or control of diseases, disorders, or conditions utilizing such compositions.

In one aspect, such compositions are ophthalmic compositions and such diseases or disorders are ophthalmic diseases or disorders.

In another aspect, the present invention provides an ophthalmic composition, comprising a pharmaceutical component having low solubility in water and at least one non-aqueous water-miscible material, such that the pharmaceutical component and the non-aqueous water-miscible material can be combined to form at least a mixture suitable for ocular administration. The non-aqueous water-miscible material is used to solubilize a pharmaceutical component that has a low aqueous solubility to enable the pharmaceutical component to be delivered to a target tissue in a therapeutically effective amount.

In still another aspect, the pharmaceutical component is solubilizable in the non-aqueous water-miscible material in an amount of at least about 0.1 mg/g. In another embodiment, the pharmaceutical component is solubilizable in the non-aqueous water-miscible material in an amount in the range from about 0.1 mg/g to about 200 mg/g.

In yet another aspect, the pharmaceutical component is a member of a group containing, for example, anti-inflammatory agents, anti-infective agents (including antibacterial, antifungal, antiviral, antiprotozoal agents), anti-allergic agents, antiproliferative agents, anti-angiogenic agents, anti-oxidants, antihypertensive agents, neuroprotective agents, cell receptor agonists, cell receptor antagonists, immunomodulating agents, immunosuppressive agents, intraocular (“IOP”) lowering agents, beta adrenoceptor antagonists, alpha-2 adrenoceptor agonists, carbonic anhydrase inhibitors, cholinergic agonists, prostaglandins and prostaglandin receptor agonists, angiotensin converting enzyme (“ACE”) inhibitors, AMPA receptor antagonists, NMDA antagonists, angiotensin receptor antagonists, somatostatin agonists, mast cell degranulation inhibitors, alpha-adrenergic receptor blockers, alpha-2 adrenoceptor antagonists, thromboxane A2 mimetics, protein kinase inhibitors, prostaglandin F derivatives, prostaglandin-2 alpha antagonists, cyclooxygenase-2 inhibitors, muscarinic agents, and combinations thereof.

In a further aspect, the pharmaceutical composition has a viscosity of between about 10 centipoises (“cp” or mPa·s) to about 10,000 cp.

In still another aspect, the non-aqueous water-miscible material can be, for example, lower alkanols (e.g., having 1 to 10, or alternatively, 1 to 6 carbon atoms; such as, ethanol), arylalkanols (e.g., having 5 to 14, or alternatively, 5 to 10 carbon atoms in the rings; such as, benzyl alcohol), polyols (e.g., having 2 to 12, or alternatively, 2 to 6 carbon atoms; such as glycerol, propylene glycol, or sorbitol), n-methylpyrrolidone, polyalkylene glycols (e.g., polyethylene glycol, propylene glycol, and the like), polyglycerin, triacetin, dimethyl acetimide, dimethyl sulfoxide, ascorbic acid, phosphate buffer vehicle systems, isotonic vehicles (e.g., boric acid, sodium chloride, sodium citrate, sodium acetate, and the like), modified vegetable oils or petroleum jelly, as well as aqueous solutions containing alkyl cellulose materials (e.g., carboxymethyl cellulose, carboxyethyl cellulose, hydroxypropylmethylcellulose, hydroxypropylethylcellulose, etc.), carbopol, polyvinyl alcohol, polyvinyl pyrrolidone, isopropyl myristate, other ophthalmic field employed, non-toxic, pharmaceutically acceptable organic and inorganic carriers, derivatives thereof, or mixtures thereof.

In a further aspect of the present invention, the pharmaceutical composition is suitable for formulation for ocular administration and can deliver a therapeutically effective amount of a pharmaceutical component when administered into, for example, the vitreous humor or into the subconjunctiva of a human or animal eye, or other posterior regions of the ocular environment.

In a still further aspect of the present invention, the composition includes at least one additive including, but not limited to, preservatives, anti-oxidants, surfactants, buffering agents, tonicity-adjusting agents, emulsifying agents, derivatives thereof, or combinations thereof.

In another aspect of the present invention, a method of preparing a pharmaceutical composition is provided that comprises providing at least one non-aqueous water-miscible material, and solubilizing in the non-aqueous water-miscible material at least one pharmaceutical component having a low aqueous solubility. The pharmaceutical component is solubilizable in the water-miscible material in an amount sufficient to provide a therapeutically effective amount of the pharmaceutical composition at or to a target tissue. In one embodiment, such target tissue is an ocular tissue.

In yet another aspect, the method of preparing such a pharmaceutical composition further comprises sterilizing said composition by; for example, sterile filtration, utilizing a filter having a pore size of at least about 0.2 micrometer or less; thermal sterilization at a temperature of at least about 150° C. for a period of at least about 25 minutes; or irradiating the mixture with gamma radiation.

In a further aspect, the present invention provides a method of treating or controlling an ocular disease, disorder, or condition. The method comprises administering a therapeutically amount of a formulation that comprises a pharmaceutical component and a non-aqueous water-miscible material to an ocular tissue in need of such treatment or control, wherein the pharmaceutical component is solubilizable in such non-aqueous water-miscible material but has a low aqueous solubility. In one embodiment, the method comprises injecting the composition into such ocular tissue. The ocular tissue can be, for example, the vitreous humor or the subconjunctiva within a human or an animal eye.

In yet a further aspect of the present invention, the ocular disease, disorder, or condition can include, but are not limited to, a posterior-segment disease or disorder. In certain embodiments, such disease or disorder is selected from the group consisting of diabetic retinopathy, diabetic macular edema, cystoid macular edema, age macular degeneration (including the wet and dry form), optic neuritis, retinitis, chorioretinitis, intermediate and posterior uveitis, choroidal neovascuralization, and combinations thereof.

These and other features and advantages of the present invention will be further understood and appreciated by those skilled in the art by reference to the following detailed description and claims.

### DETAILED DESCRIPTION OF THE INVENTION

As used herein, the term “control” also includes reduction, amelioration, alleviation, and prevention.

As used herein, the phrase “low aqueous solubility” or “low solubility in water” means solubility in water of less than 0.1 mg/g at physiological pH (about 7.4) and at about 25° C. Although compositions and methods of the present invention are particularly applicable to pharmaceutical components or compounds having such solubility, such compositions and methods are also useful in providing novel formulations of enhanced concentrations of pharmaceutical compounds, which have solubility in water in the range of less than 5 mg/g and are difficult to be formulated into compositions having therapeutically significant concentrations.

Throughout this disclosure, unless otherwise specified, concentrations of an ingredient of the composition or formulation are in weight percent.

In general, the present invention provides pharmaceutical compositions, pharmaceutical kits, and methods of treatment or control of diseases or disorders utilizing such compositions.

In one aspect, such compositions are ophthalmic compositions and such diseases or disorders are ophthalmic diseases or disorders.

In another aspect, the present invention provides an ophthalmic composition, comprising at least one pharmaceutical component and at least one non-aqueous water-miscible material, such that the pharmaceutical component and the non-aqueous water-miscible material can be combined to form at least a mixture suitable for formulation for ocular injection. The non-aqueous water-miscible material is used to solubilize a pharmaceutical component that has a low aqueous solubility to enable the pharmaceutical component to be delivered to a target tissue in a therapeutically effective amount.

In one aspect, a pharmaceutical composition of the present invention is suitable for treating ocular diseases, disorders, or conditions via ocular injection (e.g., intravitreal injection).

A variety of pharmaceutical components known within the pharmaceutical industry are suitable for use in accordance with the teachings of the present invention. Preferred pharmaceutical components are those utilized in treating ocular indications, diseases, syndromes, injuries, and the like. Additionally, although not wanting to be bound by any particular theory, Applicant believes that the present invention is particularly suited for use with pharmaceutical components that are water insoluble or poorly water-soluble, but are solubilizable in water-miscible materials. Thus, the present invention provides enhancements to the delivery, bioavailability and target tissue concentrations of such insoluble or poorly soluble pharmaceutical components.

Non-limiting examples of pharmaceutical components, including water-insoluble or poorly water soluble pharmaceutical components, especially those for use in an ocular environment according to the teachings of the present invention, include, but are not limited to, anti-inflammatory agents, anti-infective agents (including antibacterial, antifungal, antiviral, antiprotozoal agents), anti-allergic agents, antiproliferative agents, anti-angiogenic agents, anti-oxidants, antihypertensive agents, neuroprotective agents, cell receptor agonists, cell receptor antagonists, immunomodulating agents, immunosuppressive agents, IOP lowering agents, beta adrenoceptor antagonists, alpha-2 adrenoceptor agonists, carbonic anhydrase inhibitors, cholinergic agonists, prostaglandins and prostaglandin receptor agonists, angiotensin converting enzyme (“ACE”) inhibitors, AMPA receptor antagonists, NMDA antagonists, angiotensin receptor antagonists, somatostatin agonists, mast cell degranulation inhibitors, alpha-adrenergic receptor blockers, alpha-2 adrenoceptor antagonists, thromboxane A2 mimetics, protein kinase inhibitors, prostaglandin F derivatives, prostaglandin-2 alpha antagonists, cyclooxygenase-2 inhibitors, muscarinic agents, and combinations thereof.

In one embodiment, the pharmaceutical component is selected from the group consisting of anti-inflammatory agents, anti-infective agents (including antibacterial, antifungal, antiviral, antiprotozoal agents), anti-allergic agents, antiproliferative agents, anti-angiogenic agents, anti-oxidants, antihypertensive agents, neuroprotective agents, cell receptor agonists, cell receptor antagonists, immunomodulating agents, immunosuppressive agents, IOP lowering agents, and combinations thereof.

In another embodiment, the pharmaceutical component is selected from the group consisting of anti-inflammatory agents, antiproliferative agents, anti-angiogenic agents, neuroprotective agents, immunomodulating agents, IOP lowering agents, and combinations thereof.

In still another embodiment, the pharmaceutical component is selected from the group consisting of beta adrenoceptor antagonists, alpha-2 adrenoceptor agonists, carbonic anhydrase inhibitors, cholinergic agonists, and prostaglandin receptor agonists.

In a further embodiment, the pharmaceutical component is selected from the group consisting of prostaglandin agonist, beta-2 agonist, muscarinic antagonist, and combinations thereof.

In one embodiment, the pharmaceutical component comprises a fluoroquinolone having Formula I (a new-generation fluoroquinolone antibacterial agent, disclosed in U.S. Pat. No. 5,447,926, which is incorporated herein by reference).

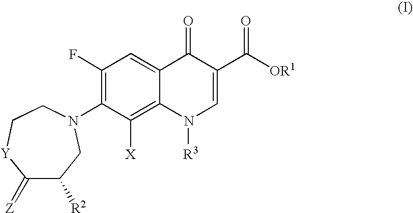

wherein R¹ is selected from the group consisting of hydrogen, unsubstituted lower alkyl groups, substituted lower alkyl groups, cycloalkyl groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, and groups that can be hydrolyzed in living bodies; R² is selected from the group consisting of hydrogen, unsubstituted amino group, and amino groups substituted with one or two lower alkyl groups; R³ is selected from the group consisting of hydrogen, unsubstituted lower alkyl groups, substituted lower alkyl groups, cycloalkyl groups, unsubstituted lower alkoxy groups, substituted lower alkoxy groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, unsubstituted C₅-C₂₄ aryloxy groups, substituted C₅-C₂₄ aryloxy groups, unsubstituted C₅-C₂₄ heteroaryloxy groups, substituted C₅-C₂₄ heteroaryloxy groups, and groups that can be hydrolyzed in living bodies; X is selected from the group consisting of halogen atoms; Y is selected from the group consisting of CH₂, O, S, SO, SO₂, and NR⁴, wherein R⁴ is selected from the group consisting of hydrogen, unsubstituted lower alkyl groups, substituted lower alkyl groups, and cycloalkyl groups; and Z is selected from the group consisting of oxygen and two hydrogen atoms.

In another embodiment, the pharmaceutical component comprises a fluoroquinolone having Formula II.

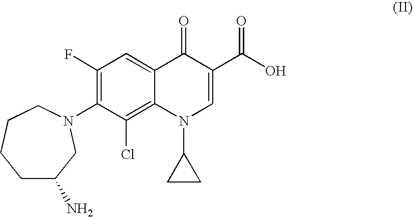

((R)-(+)-7-(3-amino-2,3,4,5,6,7-hexahydro-1H-azepin-1-yl)-8-chloro- 1 -cyclopropyl-6-fluoro-1,4-dihydro-4-oxoquinoline-3-carboxylic acid).

In still another embodiment, the pharmaceutical component comprises a glucocorticoid receptor agonist having Formulae III or IV, as disclosed in US Patent Application Publication 2006/0116396, which is incorporated herein by reference.

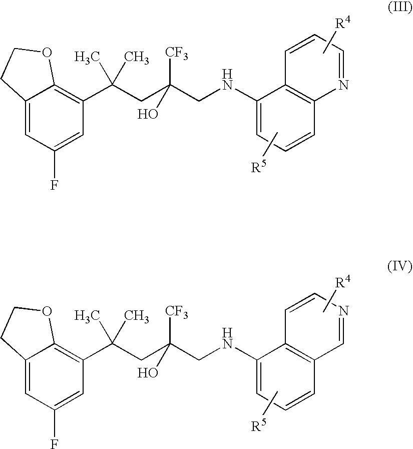

wherein R⁴ and R⁵ are independently selected from the group consisting of hydrogen, halogen, cyano, hydroxy, C₁-C₁₀ (alternatively, C₁-C₅ or C₁-C₃) alkoxy groups, unsubstituted C₁-C₁₀ (alternatively, C₁-C₅ or C₁-C₃) linear or branched alkyl groups, substituted C₁-C₁₀ (alternatively, C₁-C₅ or C₁-C₃) linear or branched alkyl groups, unsubstituted C₃-C₁₀ (alternatively, C₃-C₆ or C₃-C₅) cyclic alkyl groups, and substituted C₃-C₁₀ (alternatively, C₃-C₆ or C₃-C₅) cyclic alkyl groups.

In yet another embodiment, the pharmaceutical component comprises a glucocorticoid receptor agonist having Formula V (a species of compound having Formula III).

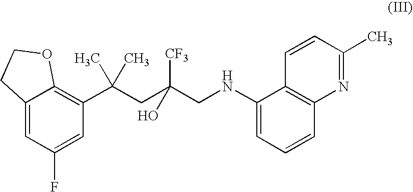

In another aspect, compositions, kits, and methodologies of the present invention are envisaged to be suitable and useful to deliver pharmaceutical components to other tissues of a human or animal. Thus, pharmaceutical components that are poorly soluble in water that can have pharmaceutical efficacy in a number of therapeutic and diagnostic arenas are applicable for use with and application of the present invention.

Non-limiting classes and examples of pharmaceutical compounds for use in arenas other than ophthalmology include, for example, hypnotic agents, sedative agents, antiepileptic agents, antipsychotic agents, neuroleptic agents, antidepressant agents, anxiolytic agents, anticonvulsant agents, antiarrhythmic agents, antihypertensive agents, hormones, nutrients, ace inhibiting agents, antidiabetic agents, antihypotensive agents, antimicotic agents, antiparkinson agents, antirheumatic agents, beta blocking agents, brochospasmolytic agents, cardiovascular agents, carotenoids, contraceptive agents, enkephalins, lipid lowering agents, lymphokines, neurologic agents, prostacyclins, psycho-pharmaceutical agents, protease inhibitors, vitamins, derivatives thereof, and combinations thereof.

Non-aqueous water-miscible materials suitable for use in the present invention include, but are not limited to, lower alkanols (e.g., having 1 to 10, or alternatively, 1 to 6 carbon atoms; such as, ethanol), arylalkanols (e.g., having 5 to 14, or alternatively, 5 to 10 carbon atoms in the rings; such as, benzyl alcohol), polyols (e.g., having 2 to 12, or alternatively, 2 to 6 carbon atoms; such as glycerol, propylene glycol, or sorbitol), n-methylpyrrolidone, polyalkylene glycols (e.g., polyethylene glycol, propylene glycol, and the like), polyglycerin, triacetin, dimethyl acetimide, dimethyl sulfoxide, ascorbic acid, phosphate buffer vehicle systems, isotonic vehicles (e.g., boric acid, sodium chloride, sodium citrate, sodium acetate, and the like), modified vegetable oils or petroleum jelly, as well as aqueous solutions containing alkyl cellulose materials (e.g., carboxymethyl cellulose, carboxyethyl cellulose, hydroxypropylmethylcellulose, hydroxypropylethylcellulose, etc.), carbopol, polyvinyl alcohol, polyvinyl pyrrolidone, isopropyl myristate, other ophthalmic field employed, non-toxic, pharmaceutically acceptable organic and inorganic carriers, derivatives thereof, or mixtures thereof.

The pharmaceutical component is solubilized, or is solubilizable, in the non-aqueous water-miscible material in an amount sufficient to obtain a therapeutically effective concentration of the pharmaceutical composition. A sufficient amount will depend upon the particular pharmaceutical component selected, the particular non-aqueous water-miscible material or materials selected, and the intended target tissue. In general, however, a sufficient amount of the pharmaceutical component is an amount of at least about 0.1 mg/g (or alternatively, at least about 1 mg/g, or at least about 2 mg/g, or at least about 5 mg/g). In another embodiment, the pharmaceutical component is solubilizable in the non-aqueous water-immiscible material in an amount in the range from about 0.1 mg/g to about 200 mg/g. Alternatively, the pharmaceutical component is solubilizable in the water-immiscible material in an amount in the range from about 0.1 mg/g to about 100 mg/g, or from about 0.1 mg/g to about 75 mg/g, or from about 0.1 mg/g to about 50 mg/g, or from about 0.1 mg/g to about 25 mg/g, or from about 0.1 mg/g to about 10 mg/g, or from about 1 mg/g to about 200 mg/g, or from about 1 mg/g to about 100 mg/g, or from about 1 mg/g to about 50 mg/g, or from about 1 mg/g to about 25 mg/g, or from about 1 mg/g to about 10 mg/g, or from about 10 mg/g to about 200 mg/g, or from about 10 mg/g to about 100 mg/g, or from about 10 mg/g to about 50 mg/g. Such solubility is measured at a physiological pH (about 7.4) and at about 25° C.

In yet another aspect, the pharmaceutical component present in the mixture at a concentration between about 0.01% (by weight) to about 50% (by weight) and the water-immiscible vehicle is present in the mixture at a concentration between about 99.99% (by weight) to about 50% (by weight) of the total weight of the mixture. In certain embodiments, the concentration of a pharmaceutical component is in the range from about 0.1% to about 25% (or alternatively, from about 0.1% to about 10%, or from about 0.1% to 5%, or from about 0.1% to about 2%, or from about 0.1% to 1%, or from about 0.5% to about 5%, or from about 0.5% to about 2%, or from about 0.2% to about 2%, or from about 0.2% to 1%) by weight. In certain other embodiments, the water-immiscible vehicle constitutes substantially the balance of the mixture (other than the presence of possible minor amounts of other additives that may be included in the mixtures).

In one aspect, the viscosity of the composition or formulation is in the range from about 10 cp to about 10,000 cp. Alternative the viscosity of the composition or formulation is in the range from about 10 cp to about 5,000 cp, or from about 10 cp to about 2,000 cp, from about 10 cp to about 1,000 cp.

In one or more embodiments of the present invention, the mixture can also include one or more additives, including, but not limited to, preservatives, non-ionic tonicity-adjusting agents, viscosity-modifying agents, solubility-enhancing agents, and combinations thereof.

Non-limiting examples of preservatives include benzalkonium chloride (BAK”), quaternary ammonium compounds (e.g., polyquat-1, polyquat-10), hydrogen peroxide, urea hydrogen peroxide, sorbic acid/EDTA (ethylenediamine tetraacetic acid), p-hydroxybenzoic acid esters, polyhexamethylene biguanide (“PHMB”), phenylethyl alcohol, ethylparaben, and methylparaben. These agents may be present in individual amounts of from about 0.001 to about 2% by weight (preferably, about 0.01% to about 1% by weight).

A viscosity-modifying compound can be designed to facilitate the administration of the composition into the subject or to promote the bioavailability in the subject for the intended time period of treatment. A viscosity-modifying compound can be a low or high molecular weight material, depending on the viscosity of the water-immiscible carrier used. A non-limiting example of a low molecular weight viscosity-modifying agent is a medium-chain triglyceride (“MCT”), wherein the fatty acyl moiety comprises 4-12 carbon atoms. A viscosity-modifying compound can be a pharmaceutically acceptable polymer of suitable molecular weight and may be chosen so that the composition is not readily dispersed after being administered into the vitreous. Such compounds may enhance the viscosity of the composition, and include, but are not limited to: long-chain triglycerides (“LCT,” wherein the fatty acyl moiety has more than 12, preferably more than 18, and more preferably more than 22, carbon atoms), water-immiscible acrylic ester polymers, polysiloxanes, and water-immiscible polypeptides.

A non-limiting example of solubility-enhancing agents is beta-cycodextrin.

In one aspect, the pharmaceutical composition can be a sustained-release, controlled release, or extended release solution or composition that releases the pharmaceutical component over a period of time. In one embodiment, the pharmaceutical composition can release the pharmaceutical component over a period of 8 hours or longer. In another embodiment, the pharmaceutical composition can release the pharmaceutical component over a period of 12 hours or longer. In another preferred embodiment, the pharmaceutical composition can release the pharmaceutical component over a period of 24 hours or longer. In another embodiment, the pharmaceutical composition can release the pharmaceutical component over a period of 2, 3, 4, 5, 6, or 7 days or longer. In another preferred embodiment, the pharmaceutical composition can release the pharmaceutical component over a period of 2, or 4 weeks or longer.

In one aspect, a composition of the present invention is formulated for topical administration. In one embodiment, such a composition is formulated for topical administration to the anterior segment of the eye, such as to the anterior ocular surface, for treating or controlling an anterior-segment disease, disorder, or condition.

Alternatively, the mixture can be formulated for injection into an ocular environment, including, but not limited to, the vitreous cavity or the subconjunctiva of an eye within a human or an animal. The mixture can be formulated for ocular injection according to known methods and principles, and then injected using an injection delivery device such as an appropriately gauged needle; for example, 25-30 gauge needle.

Optionally, before the mixture is injected into an ocular environment, the mixture can be sterilized. Suitable methods of sterilization include, but are not limited to, sterile filtration, thermal sterilization, and gamma irradiation. Where sterile filtration is selected, one suitable method of sterile filtration can utilize a filter having a pore size of at least about 0.2 micrometer or less. Where thermal sterilization is selected, one suitable method of thermal sterilization can include sterilizing the mixture at a temperature of at least about 150° C. for a period of at least about 25 minutes. Where gamma irradiation is selected, one suitable method can include exposure of the compositions of the present invention to gamma rays at a level of from about 2.5 Mrad to about 3.5 Mrad.

As noted above, another aspect of the present invention involves a method of treating an ocular disease, disorder, or condition. The method includes administering at least one mixture comprising at least one pharmaceutical component and at least one water-immiscible material into an ocular environment. The mixture can be used to treat an ocular disease, disorder, or condition including, but not limited to, diabetic retinopathy, diabetic macular edema, cystoid macular edema, age macular degeneration (including the wet and dry form), optic neuritis, retinitis, chorioretinitis, intermediate and posterior uveitis, choroidal neovascuralization, and combinations thereof.

In another aspect, a composition of the present invention including an appropriate pharmaceutical component is used to treat or control a ocular diseases, conditions, or disorders of the anterior segment including anterior uveitis (including iritis and iridocyclitis), keratitis, conjunctivitis, keratoconjunctivitis (including vernal keratoconjunctivitis (or “VKC”) and atopic keratoconjunctivitis), corneal ulcer, corneal edema, sterile corneal infiltrates, anterior scleritis, episcleritis, blepharitis, and post-operative (or post-surgical) ocular inflammation resulting from procedures such as photorefractive keratectomy, cataract removal surgery, intraocular lens (“IOL”) implantation, laser-assisted in situ keratomileusis (“LASIK”), conductive keratoplasty, and radial keratotomy.

The non-aqueous water-miscible material and the pharmaceutical component can be combined to form any suitable mixture, including, but not limited to, a water-miscible solution, a semi-solid, or a suspension. In another embodiment, the water-miscible solution can further be added to a hydrophobic medium and the total can be formed into a stable emulsion. For example, the mixture can be a suspension containing particles of the pharmaceutical component in the water-miscible material. In various embodiments of the present invention, the particles of the pharmaceutical component have a particle size of between about 0.01 μm to about 1 μm in diameter. In another embodiment, the particle size is between about 0.05 μm to about 0.5 μm in diameter.

Although not wanting to be bound by any particular theory, Applicant believes that the non-aqueous water-miscible material as a drug delivery vehicle of the present invention can address one or more of the challenges described herein regarding the delivery of pharmaceutical components to target tissues within the ocular environment.

For example, solubilization of a pharmaceutical component that has a low solubility in an aqueous medium can have a higher solubility in a nonaqueous water-miscible material. Such increased solubility can enhance the availability of that pharmaceutical component or component particles at, in, or near those target tissues, and thereby enhance the component's concentration at, in, or near the target tissues.

In some instances, the amount or dose of the pharmaceutical component can be completely soluble in the non-aqueous water-miscible material such that the entire amount or dose is delivered as a water-miscible solution to the desired ocular environment. In other instances, the pharmaceutical component can be delivered as a suspension, yet because of the higher solubility in the non-aqueous water-miscible delivery vehicle of the present invention, the concentration of the pharmaceutical component in the fluid phase of the composition can be high and thus, a more significant concentration of the pharmaceutical component is available at or near the target tissue.

An additional advantage of using a water-miscible material is the improved potential for the bioavailability of particles. As the water-miscible material dissipates, or as the ocular fluid (such as tear or vitreous humor) penetrates the composition droplet or injection bolus, very small particles of the pharmaceutical component are exposed. Under most conditions, smaller particles of a pharmaceutical component have higher bioavailability than larger particles. An added advantage of smaller particles is that they are less likely to migrate into the visual axis and occlude vision unlike conventional ocular compositions such as an ointment or ocular injectable dispersion.

The following examples further illustrate the present invention and are not to be construed as limiting the invention or scope of the specific procedures or compositions described herein.

### EXAMPLE 1

### Water/Tear Miscible Solution Formulation

5% triacetin
5% polyethylene glycol 400
5% propylene glycol
0.1% EDTA disodium
0.15 sodium ascorbate
0.1% tocophersolan (“TPGS”)
0.5% phenylethyl alcohol
q.s. compound having Formula II
q.s. NaOH (IN solution) to adjust pH to 5.5-6
q.s. water

Add all the components (except NaOH) to the water phase. Mix at high speed for 10-30 minutes. Adjust the pH to 5.5-6. The solution is useful for treating ocular bacterial infection.

### EXAMPLE 2

### Water/Tear Miscible Solution Formulation

5% propylene glycol
1% α-tocopherol
0.1% PHMB
q.s. polyethylene glycol 400
q.s. compound having Formula V
NaOH (1N solution) for pH adjustment

Add all components (except NaOH) to a sterilized vessel. Mix thoroughly for 10-30 minutes. Adjust pH to 5.5-6. The solution is useful for treating ocular inflammation.

### EXAMPLE 3

### Water/Tear Miscible Suspension Formulation

5% triacetin
5% polyethylene glycol 400
5% propylene glycol
0.1% EDTA disodium
0.15 sodium ascorbate
0.1% tocophersolan (“TPGS”)
0.5% phenylethyl alcohol
q.v. celecoxib (also known under the tradename Celebrex®, a COX-2 inhibitor)
q.s. NaOH (1N solution) to adjust pH to 5.5-6
q.s. water

Add all the components (except the drug and NaOH) to the water phase. Mix at high speed for 10-30 minutes. Adjust the pH to 5.5-6. Add the desired amount of drug substance to a small portion of the water phase such that the drug concentration is 100-500 mg/mL. Use wet milling to reduce the average particle size of the drug substance to 10 micrometers or less. Dilute this milled suspension to the desired drug concentration with additional water phase. The suspension is useful for treating ocular inflammation.

### EXAMPLE 4

### Water/Tear Miscible Solution Containing Cyclodextrin

5% triacetin
5% polyethylene glycol 400
1% beta-cyclodextrin
0.1% EDTA disodium
0.1% sodium ascorbate
0.1% tocophersolan (“TPGS”)
0.2% phenylethyl alcohol
q.s. brimonidine to saturate the formulation
q.s. NaOH (1N solution) to adjust pH to 5.5-6
q.s. water

Add all the components (except NaOH) to the water phase. Adjust the pH to 5.5-6. Intravitreal administration of this solution is useful for providing ocular neuroprotection.

### EXAMPLE 5

### Water/Tear Miscible Solution Containing Surfactant

5% triacetin
5% polyethylene glycol 400
5% propylene glycol
1% PEG-35 castor oil (Cremophor EL)
0.1% EDTA disodium
0.1% sodium ascorbate
0.1% tocophersolan (“TPGS”)
0.25% phenylethyl alcohol
q.s. moxifloxacin to saturate the formulation
q.s. NaOH (1N solution) to adjust pH to 6-6.5
q.s. water

Add all the components (except NaOH) to the water phase. Adjust the pH to 6-6.5. This solution is useful for topical administration to treat ocular bacterial infection.

### EXAMPLE 6

### Viscosity-Enhanced Water/Tear Miscible Solution Formulation Containing Surfactant

5% triacetin
5% polyethylene glycol 400
5% propylene glycol
1% PEG-3 5 castor oil (Cremophor EL)
0.2% Carbomer 980
0.1% EDTA disodium
0.1% sodium ascorbate
0.1% tocophersolan (“TPGS”)
0.25% phenylethyl alcohol
q.s. cyclosporine A to saturate the formulation
q.s. NaOH (1N solution) to adjust pH to 6-6.5
q.s. water

Combine triacetin, PEG 400, propylene glycol PEG-35 castor oil, phenylethyl alcohol, and cyclosporine A together. Mix to dissolve the drug. Add the remaining components to the water and disperse the Carbomer 980 in the water under high shear until it dissolves. Add the portion containing cyclosporine A to the portion containing Carbomer 980 and mix until uniformity is achieved. Adjust the pH to 6-6.5. This solution is useful to treat or relieve the dry eye syndrome.

### EXAMPLE 7

### Water Miscible Solution Formulation Not Containing Water

5% DMSO
0.1% polysorbate 80
94.9% polyethylene glycol 400
q.s. latanoprost (prostaglandin analog) to saturate the formulation

Mix all the components (except latanoprost) together until uniformity is achieved. Add latanoprost to the mixture while mixing until saturation. This formulation may be useful as a starting material for further preparation of a composition for lowering IOP in a patient.

The invention has now been described in such full, clear, concise and exact terms as to enable any person skilled in the art to which it pertains, to practice the same. It is to be understood that the foregoing describes preferred embodiments and examples of the invention and that modifications may be made therein without departing from the spirit or scope of the invention as set forth in the claims. Moreover, while particular elements, embodiments and applications of the present invention have been shown and described, it will be understood, of course, that the present invention is not limited thereto since modifications can be made by those skilled in the art without departing from the scope of the present disclosure, particularly in light of the foregoing teachings and appended claims. Moreover, it is also understood that the embodiments as described above are merely for illustrative purposes and not intended to limit the scope of the invention, which is defined by the following claims as interpreted according to the principles of patent law, including the Doctrine of Equivalents. Further, all references cited herein are incorporated in their entirety.

What is claimed is:

1. A method of preparing a pharmaceutical composition comprising the steps of:
a) providing a non-aqueous water-miscible material;
b) solubilizing in the non-aqueous water-miscible material a pharmaceutical component having a low aqueous solubility,
wherein the pharmaceutical component is solubilizable in the non-aqueous water-miscible material in an amount sufficient to obtain a therapeutically effective concentration of the pharmaceutical composition; and
wherein the non-aqueous water-miscible material is present in the composition in an amount sufficient to deliver a therapeutically effective amount of the pharmaceutical component when the composition is administered into a target tissue.

2. The method of claim 1, wherein the non-aqueous water-miscible material is selected from the group consisting of alkanols having 1-6 carbon atoms, arylalkanols having 5 to 10 carbon atoms, polyols having 2 to 6 carbon atoms, n-methylpyrrolidone, polyalkylene glycols, polyglycerin, triacetin, dimethyl acetimide, dimethyl sulfoxide, ascorbic acid, phosphate buffer vehicle systems, isotonic vehicles, modified vegetable oils or petroleum jelly, as well as aqueous solutions containing alkyl cellulose materials, carbopol, polyvinyl alcohol, polyvinyl pyrrolidone, isopropyl myristate, other ophthalmic field employed, non-toxic, pharmaceutically acceptable organic and inorganic carriers, derivatives thereof, and mixtures thereof.

3. The method of claim 1, wherein the non-aqueous water-miscible material is mixed with water to provide a mixture of aqueous and non-aqueous materials.

4. The method of claim 1, wherein the sufficient amount of the pharmaceutical component solubilizable in the non-aqueous water-miscible material is an amount of at least about 0.1 mg/g.

5. The method of claim 1, wherein the pharmaceutical component is a member selected from the group consisting of: anti-inflammatory agents, anti-infective agents, anti-allergic agents, antiproliferative agents, anti-angiogenic agents, anti-oxidants, antihypertensive agents, neuroprotective agents, cell receptor agonists, cell receptor antagonists, immunomodulating agents, immunosuppressive agents, IOP lowering agents, beta adrenoceptor antagonists, alpha-2 adrenoceptor agonists, carbonic anhydrase inhibitors, cholinergic agonists, prostaglandins and prostaglandin receptor agonists, angiotensin converting enzyme (“ACE”) inhibitors, AMPA receptor antagonists, NMDA antagonists, angiotensin receptor antagonists, somatostatin agonists, mast cell degranulation inhibitors, alpha-adrenergic receptor blockers, alpha-2 adrenoceptor antagonists, thromboxane A2 mimetics, protein kinase inhibitors, prostaglandin F derivatives, prostaglandin-2 alpha antagonists, cyclooxygenase-2 inhibitors, muscarinic agents, and combinations thereof.

6. The method of claim 1, further comprising the step of sterilizing the composition.

7. A pharmaceutical composition comprising:
a pharmaceutical component having a low aqueous solubility; the pharmaceutical component being present in the composition in an amount that is therapeutically effective when the composition is administered to or into a target tissue; and
a non-aqueous water-miscible material in an amount sufficient to solubilize said therapeutically effective amount of the pharmaceutical component.

8. The pharmaceutical composition of claim 7, wherein the pharmaceutical component is solubilized in the non-aqueous water-miscible material in an amount of at least about 0.1 mg/g.

9. The pharmaceutical composition of claim 7, wherein the pharmaceutical component is selected from the group consisting of anti-inflammatory agents, anti-infective agents, anti-allergic agents, antiproliferative agents, anti-angiogenic agents, anti-oxidants, antihypertensive agents, neuroprotective agents, cell receptor agonists, cell receptor antagonists, immunomodulating agents, immunosuppressive agents, IOP lowering agents, beta adrenoceptor antagonists, alpha-2 adrenoceptor agonists, carbonic anhydrase inhibitors, cholinergic agonists, prostaglandins and prostaglandin receptor agonists, angiotensin converting enzyme (“ACE”) inhibitors, AMPA receptor antagonists, NMDA antagonists, angiotensin receptor antagonists, somatostatin agonists, mast cell degranulation inhibitors, alpha-adrenergic receptor blockers, alpha-2 adrenoceptor antagonists, thromboxane A2 mimetics, protein kinase inhibitors, prostaglandin F derivatives, prostaglandin-2 alpha antagonists, cyclooxygenase-2 inhibitors, muscarinic agents, and combinations thereof.

10. The pharmaceutical composition of claim 9, wherein the composition further includes water mixed with the non-aqueous water-miscible material.

11. The pharmaceutical composition of claim 10, wherein the non-aqueous water-miscible material is selected from the group consisting of alkanols having 1 to 10 carbon atoms, arylalkanols having 5 to 14 carbon atoms, polyols having 2 to 12 carbon atoms, n-methylpyrrolidone, polyalkylene glycols, polyglycerin, triacetin, dimethyl acetimide, dimethyl sulfoxide, ascorbic acid, phosphate buffer vehicle systems, isotonic vehicles, modified vegetable oils or petroleum jelly, as well as aqueous solutions containing alkyl cellulose materials, carbopol, polyvinyl alcohol, polyvinyl pyrrolidone, isopropyl myristate, other ophthalmic field employed, non-toxic, pharmaceutically acceptable organic and inorganic carriers, derivatives thereof, and mixtures thereof.

12. The pharmaceutical composition of claim 9, wherein the non-aqueous water-miscible material is compatible with ocular tissue.

13. The pharmaceutical composition of claim 11, further comprising an additive selected from the group consisting of preservatives, anti-oxidants, surfactants, buffering agents, tonicity-adjusting agents, emulsifying agents, derivatives thereof, and combinations thereof.

14. The pharmaceutical composition of claim 10, wherein the composition has a viscosity of between about 10 cp to about 10,000 cp.

15. The pharmaceutical composition of claims 7, wherein said pharmaceutical component comprises a compound having Formula V.

16. The pharmaceutical composition of claims 7, wherein said pharmaceutical component comprises a compound having Formula II.

17. A method of treating or controlling an ocular disease, disorder, or condition, the method comprising:
administering to or into an ocular environment a therapeutically effective amount of a mixture comprising a pharmaceutical component having low aqueous solubility solubilized in a non-aqueous water-miscible material, to treat or control said disease, disorder, or condition.

18. The method of claim 17, wherein the ocular disease, disorder, or condition is selected from the group consisting of diabetic retinopathy, diabetic macular edema, cystoid macular edema, age macular degeneration (including the wet and dry form), optic neuritis, retinitis, chorioretinitis, intermediate and posterior uveitis, choroidal neovascuralization, anterior uveitis (including iritis and iridocyclitis), keratitis, conjunctivitis, keratoconjunctivitis (including vernal keratoconjunctivitis (or “VKC”) and atopic keratoconjunctivitis), corneal ulcer, corneal edema, sterile corneal infiltrates, anterior scleritis, episcleritis, blepharitis, and post-operative (or post-surgical) ocular inflammation resulting from procedures such as photorefractive keratectomy, cataract removal surgery, intraocular lens (“IOL”) implantation, laser-assisted in situ keratomileusis (“LASIK”), conductive keratoplasty, radial keratotomy, and combinations thereof.

19. The method of claim 17, wherein said administering is carried out by topical administration to treat or control an anterior-segment ocular disease, disorder, or condition.

20. The method of claim 17, wherein said administering is carried out by intravitreal administration to treat or control a posterior-segment ocular disease, disorder, or condition.
